(19) United States
(12) Patent Application Publication
(10) Pub. No.: US 2023/0031159 A1
(43) Pub. Date: Feb. 2, 2023

(54) WEARABLE DEVICE WITH SENSORS FOR HEALTH AND SAFETY MONITORING

(71) Applicant: Luxembourg Institute of Science and Technology (LIST), Esch-sur-Alzette (LU)

(72) Inventor: Djamel Khadraoui, Terville (FR)

(21) Appl. No.: 17/789,540

(22) PCT Filed: Dec. 29, 2020
(86) PCT No.: PCT/EP2020/087971

§ 371 Date: Jun. 28, 2022

(30) Foreign Application Priority Data
Dec. 30, 2019 (LU) LU101577

(51) Int. Cl. A61B 5/00; A61B 5/01

(52) U.S. Cl. CPC A61B 5/7282; A61B 5/746; A61B 5/0008; A61B 5/01; A61B 5/6802; A61B 2562/0271; A61B 2560/0242

(57) ABSTRACT

A monitoring process, a user health and/or safety monitoring process, the monitoring process including the steps: providing a sensor set including at least one sensor; obtaining a monitoring configuration from a memory element. The monitoring configuration including: a sensor configuration associated with the sensor set and a body profile associated with a subject body and comprising reference data. The monitoring process further providing, in a memory element, measurement data relating to said subject body, said measurement data being acquired by said sensor set; computing, with computing means, a difference between the measurement data and the reference data; comparing, with comparison means, the difference and a predefined condition; changing, and with changing means, the monitoring configuration depending on the result of the comparing step.

### TECHNICAL FIELD

The invention lies in the field of monitoring by means of a wearable device. More precisely, the invention provides a monitoring process of a subject body by means of sensors. The invention also provides a corresponding computer program and a wearable device for health and/or safety of a user.

### BACKGROUND OF THE INVENTION

A wearable device provides, by means of its sensors, real time monitoring data related to safety and health of the user. This comfortable and lightweight system is worn directly by the user, at a close location from a relevant body area. Then, accurate data is obtained all day long, or at least during working hours. In case of anomaly, a warning signal is sent upon detection and a safety measure it triggered.

However, the accuracy of a wearable device, and the trustworthiness rely on the correct functioning of the sensors, and of the configuration of the wearable device. Where the sensors provide erroneous data, a false warning situation may be communicated, or a real dangerous situation risks not to be communicated. Consequently, the user is exposed to a hazardous situation.

The document US2009/275805 A1 discloses an on-demand help/in-service for a medical device. The medical equipment includes a thermometer probe for making an oral temperature measurement. A temperature measurement that is thought to be too low, or a fluctuating temperature can be displayed as visible numbers either slightly “grayed out” under the assistance screen, or in another window within or adjacent to the displayed assistance screen. Then, the medical device increases safety by changing the display configuration. However, the medical device does not provide a satisfying safety level since the temperature analysis may rely on fallacious data.

The document US2013/317753 A1 discloses a system, a method, and an apparatus for electronic patient care. If the BP monitor 15 indicates a blood pressure below a predetermined acceptable range, the monitoring client 1 may be programmed to instruct the infusion pump 7 to stop the infusion, and it can transmit an urgent notification 12 to the health care provider(s)′ monitoring clients 11. Nevertheless, the comparison of the blood pressure with respect to the acceptable range is exposed to defects. Accordingly, there is still room for improving safety.

The document US2007/056582 A1 discloses methods and devices for relieving stress. The methods identify RSA waves during respiration which provide a subject with real-time RSA wave information. The methods also detect and correct erroneous data relating to RSA waves. Yet, the device does not fulfil safety requirements since it only corrects data as such. The device fails to detect error sources. It does not provide an adequate correction when it generates a falacious analysis framework which further distorts said erroneous data.

### Technical Problem to be Solved

It is an objective of the invention to present a monitoring process, which overcomes at least some of the disadvantages of the prior art. In particular, it is an objective of the invention to present a monitoring process which improves the safety.

### SUMMARY OF THE INVENTION

According to a first aspect of the invention it is provided a monitoring process, notably a user health and/or safety monitoring process, the monitoring process comprising the steps: providing a sensor set including at least one sensor; obtaining a monitoring configuration from a memory element, the monitoring configuration including: a sensor configuration associated with the sensor set, a body profile associated with a subject body and comprising reference data; providing, in a memory element, measurement data relating to said subject body, said measurement data being acquired by said sensor set; computing, with computing means, a difference between the measurement data and the reference data; comparing, with comparison means, the difference and a predefined condition; changing, with changing means, the monitoring configuration depending on the result of step comparing.

Preferably, provided that at step comparing the difference meets the predefined condition, the monitoring process further comprises the steps: generating, with generating means, a configuration change signal, transmitting, with transmitting means, a configuration change signal, and receiving, with receiving means, the configuration change signal, at step changing the monitoring configuration is changed in accordance with the configuration change signal.

Preferably, the receiving means are comprised in the sensor set.

Preferably, the predefined condition comprises a predefined threshold, at step comparing the difference meets the predefined condition provided that the measurement data diverges from the reference data by, at least, the predefined threshold.

Preferably, the reference data comprises a reference range, and the body profile comprises a rest state with a rest value within the reference range.

Preferably, if the measurement data comprises a piece of data in the reference range, and the difference does not meet the predefined condition, the monitoring process comprises a step generating a warning signal indicating that the subject body is in an unhealthy state or an unsafe state.

Preferably, the at least one sensor is a first sensor, at step providing measurement data the first sensor is enabled, and at step changing the first sensor is disabled.

Preferably, the sensor set further comprises a second sensor, at step providing measurement data the second sensor is disabled, and at step changing the second sensor is enabled.

Preferably, step providing measurement data comprises a sub-step measuring said measurement data by means of the sensor set.

Preferably, the reference data is a first reference data, the difference is a first difference, at step obtaining the monitoring configuration, the body profile is a first body profile of a profile library further comprising a second body profile with a second reference data, step changing optionally comprising a sub-step replacing the first body profile by the second body profile, the monitoring process further comprising a step computing a second difference between the measurement data and the second reference data; the first difference meeting the predefined condition and the second difference does not meet said predefined condition.

Preferably, the predefined condition comprises a first environment threshold, a first body information threshold or a body reference range, which are optionally associated with the first environment threshold; the monitoring process comprising a step providing environment information and a body information, and comprising said step changing provided that said environment information exceeds the first environment threshold and the body information is below the first body information threshold or the body reference range.

Preferably, the predefined condition comprises a first temperature threshold and a second temperature threshold, optionally associated with the first temperature threshold; the monitoring process comprising a step providing a body temperature and an environment temperature, and comprising said step changing provided that the environment temperature exceeds the first temperature threshold and the body temperature is below the second temperature threshold. Preferably, the predefined condition comprises a first hygrometry threshold and a first perspiration threshold, optionally associated with the first hygrometry threshold; the monitoring process comprising a step providing a body perspiration information and an environment hygrometry, and comprising said step changing provided that the environment hygrometry exceeds the first hygrometry threshold and the body perspiration information is below the first perspiration threshold.

Preferably, the predefined condition comprises a third temperature threshold and a second perspiration threshold, optionally associated with the third temperature threshold, the monitoring process comprising a step providing an environment temperature and a body perspiration information, and comprising said step changing provided that the environment temperature exceeds the third temperature threshold and the body perspiration information is below the second perspiration threshold.

Preferably, the predefined condition comprises a second body information threshold and a motion threshold, the monitoring process further comprises a step tracking motion information of the subject body, and a step providing a body information such as the heart rate, the subject body temperature, the subject body perspiration; the monitoring process comprising said step changing provided that the body information is below the second body information threshold and the motion information exceeds the motion threshold.

Preferably, at step computing the difference, weight parameters are applied to pieces of data of the measurement data, said weight parameters being computed by means of at least one of the following: a subject body temperature, a subject body physical activity, and a subject body perspiration information.

Preferably, the subject body is a first subject body, the body profile is a first body profile, the measurement data is a first measurement data, the monitoring process further comprising a step identifying a neighbour subject body with a neighbour sensor set including at least one sensor; a neighbour body profile defined by a neighbour reference data, the distance between the first subject body and the neighbour subject body being smaller than a threshold distance; a step acquiring neighbour measurement data relating to the neighbour subject body; a step computing a neighbour difference between the neighbour measurement data and the neighbour reference data; the monitoring process comprising said step changing provided that the neighbour difference does not meet the predefined condition.

Preferably, the first body profile and the neighbour body profile are identical, the first subject body and the neighbour subject body comprising different sensor configurations; or the first subject body and the neighbour subject body comprises identical sensor configurations, and the first body profile differs from the neighbour body profile.

Preferably, at step changing the monitoring configuration is changed into an updated monitoring configuration and the subject body is in a predefined area, the monitoring process further comprising a step changing a monitoring configuration, into said updated monitoring configuration, of another subject body which is in said predefined area.

Preferably, the monitoring process comprises a step capturing or obtaining environment information, at step comparing the difference between the measurement data and the reference data is computed with a neuronal network model adapted for detecting anormal monitoring configurations by means of said environment information.

Preferably, at step changing, the process comprises a step capturing or obtaining environment information, said environment information feeding a learning database of anormal monitoring configurations, the process further comprising a step training a neuronal network by means of the learning database in order to compute said neuronal network model.

Preferably, the subject body is a first subject body which is part of a group of subject bodies, each subject body being associated with a sensor set including at least one sensor; a monitoring configuration with a sensor configuration associated and a body profile comprising a reference data; the monitoring process comprising a step defining, within said group, a first sub group of subject bodies which comprise similar or identical monitoring configurations, said first sub group being defined by means of a neural network model configured for defining sub groups on the basis of their measurement data, the first subject body being part of said first sub group.

Preferably, at step changing the monitoring configuration of the first subject body is changed into an updated monitoring configuration, the monitoring process comprises a step changing monitoring configurations of the other subject bodies of the first sub group into said updated monitoring configuration.

Preferably, the monitoring process comprises a step updating the monitoring configuration into an updated monitoring configuration; the monitoring process repeating steps providing, computing and comparing while a predefined period and/or a predefined number of iterations during which the difference does not meet the predefined condition; then the monitoring process further comprises a step changing the monitoring configuration of a third subject body into said updated monitoring configuration.

Preferably, the sensor set comprises at least one biometric sensor adapted for measuring biometric data of said subject body.

Preferably, the sensor set comprises at least one environment sensor adapted for acquiring an information of the environment of the subject body, said at least one environment sensor notably comprising at least one of the following: a camera, a microphone, a thermometer, a hygrometer, or a manometer adapted for measuring the environment pressure.

Preferably, the sensor set further comprises a remote sensor at distance from the subject body, said remote sensor notably being fixed to a ground-based infrastructure with respect to which the subject body moves during the monitoring process.

Preferably, at step providing measurement data, the at least one sensor is physically in contact of the subject body and is adapted for measuring at least one of the following: a temperature of the subject body, a humidity level on the subject body, a heart rate of the subject body, and a blood pressure of the subject body; the humidity level notably being a perspiration on the skin of the subject body.

Preferably, the body profile comprises at least one of the following: the weight of the subject body, the size of the subject body, the age of the subject body, and an average physical activity of the subject body.

Preferably, the subject body is a human subject body.

Preferably, the monitoring process is a health monitoring process and/or a user monitoring process and/or a sensor monitoring process, and/or a subject body monitoring process.

Preferably, at step changing the body profile is changed, and/or the sensor set is changed, notably replaced, and/or the sensor configuration is changed.

Preferably, at step changing at least one sensor from the sensor set is disabled.

Preferably, the monitoring process further comprises a step computing a first difference between the measurement data and the reference data, at step obtaining the reference data is a first reference data, and the second body profile comprises a second reference data, a second difference between the reference data and the measurement data is smaller than the predefined threshold or outside a predefined range compelling the execute said step changing.

Preferably, the monitoring process comprises a step computing a comparison information, said comparison information comprising a mathematical formula with at least one of the following: a polynomial, a ratio, and a difference.

Preferably, the monitoring process comprises a temperature analysis in order to mitigate a body temperature on the basis of an environment temperature, if the environment temperature exceed a first temperature threshold and the body temperature is comprised in a temperature range, then the process does not execute the steps generating and changing.

Preferably, the data is correlated with the environment of the user at the moment the user data is measured and/or obtained.

Preferably, at step comparing the predefined threshold is defined in relation with environment data, and/or at step obtaining the reference data is defined in relation with environment data.

Preferably, the measurement data comprises biometric data and/or physiological data.

Preferably, the process comprises a step assessing whether the measurement data differs of the reference data from a predefined threshold, the monitoring process comprising said step changing depending on whether the measurement data differs of the reference data from the predefined threshold.

Preferably, the monitoring process comprises a step adapting the predefined threshold on the basis of a body temperature and a body perspiration.

Preferably, the sensor set comprises an indoor sensor.

Preferably, the monitoring process comprises the steps generating and/or changing if an environment information exceeds a first environment threshold and a body information is outside a body information range, optionally associated with the first environment threshold, else the monitoring process does not comprise the steps generating and/or changing.

Preferably, the monitoring process further comprises a step recording or obtaining physical activity data of the body, at step comparing the measurement data being adapted by means of the physical activity data.

Preferably, if at step comparing the difference does not meet the predefined condition; the monitoring process starts again the step providing measurement data.

Preferably, the monitoring process comprises a step mitigating the measurement data by means of at least one of the following: a subject body temperature, a subject body physical activity, and a subject body perspiration information.

Preferably, the monitoring process comprises a sub step aggregating data.

Preferably, the monitoring process comprises a sub step normalizing data, in order to prepare data to sub step aggregating.

Preferably, the predefined conditions are part of the monitoring configuration; at step changing, the predefined conditions are changed with changing means, depending on the result of step comparing.

Preferably, the monitoring process, optionally step comparing, comprises a sub step detecting an anomaly when the difference diverges and/or does not meet the predefined condition.

Preferably, the monitoring process further comprises a step identifying specific situations and/or first anomalies in the predefined area.

Preferably, the subject bodies of the first sub group each comprise the same or a similar sensor set. Preferably, the wearable device weights at most: 15 kg; or 5 kg; or 1 kg, 200 g or 100 g.

Preferably, the size of the wearable device is of at most: 20 cm, or 10 cm, or 7 cm. Then, the wearable device remains comfortable for a daily use. The motion freedom is kept.

Preferably, the wearable device is connected by means of a wireless module.

Preferably, the wearable device is implanted in the subject body.

Preferably, the step of comparing comprises comparing the difference with respect to the predefined condition.

Preferably, at the step of changing, the monitoring configuration is changed into an updated monitoring configuration.

Preferably, at the step of changing, the body profile is changed into an updated body profile.

Preferably, at the step of changing, the sensor configuration is changed into an updated sensor configuration.

Preferably, the process comprises a step monitoring the monitoring configuration depending on the updated monitoring configuration; and/or the updated sensor configuration, and/or the updated body profile.

Preferably, the step of changing the monitoring configuration may be and/or comprise a step of correcting, with correcting means, the monitoring configuration.

It is another aspect of the invention to provide a user monitoring process, notably a user health and/or safety monitoring process, the monitoring process comprising the steps: providing a set of wearable devices; each wearable device comprising at least one sensor such as biometric sensor, the set comprising a first wearable device; obtaining a monitoring configuration from memory means, the monitoring configuration including: a sensor configuration associated with each wearable device, and a reference data; obtaining measurement data from the at least one sensor of the first wearable device; computing, with computing means, a difference between the measurement data and the reference data; comparing, with comparison means, the difference and a predefined condition; detecting; with detection means; an anomaly; changing, with changing means, the monitoring configuration depending on the result of step comparing.

Preferably, the monitoring process further comprises changing the monitoring configuration of the other wearable devices of the set, and applying the updated monitoring configuration of the first wearable devices to the other wearable devices of the set.

Preferably, the set of wearable devices is a first set of first wearable devices, the sensors beings first sensors, the monitoring process further comprising a step providing a second set of second wearable devices, each second wearable device comprising at least one second sensor.

Preferably, the anomaly is a first anomaly, the process further comprising a step detecting a second anomaly in the second set, and further comprises changing the monitoring configuration of at least one wearable device from the second set into a second updated monitoring configuration; the monitoring process further comprising a step applying the second updated monitoring configuration to the wearable devices of the first set.

It is another aspect of the invention to provide a sensor monitoring process, the monitoring process comprising the following steps: providing a sensor set including at least one sensor, notably equipping a wearable device equipping a user or a user body, obtaining a monitoring configuration from memory means, the monitoring configuration including: a sensor configuration associated with the sensor set and/or a body profile associated with a subject body and comprising reference data; providing, in memory means, measurement data for instance relating to said subject body, said measurement data being acquired by said sensor set; computing, with computing means, a difference between the measurement data and the reference data; comparing, with comparison means, the difference and a predefined condition; changing, with changing means, the sensor configuration depending on the result of step comparing.

It is another aspect of the invention to provide a health and/or safety monitoring process for a user wearing a wearable device, the process comprising the following steps: providing a sensor set including at least one sensor, the sensors set being distributed amongst wearable devices worn by said user, obtaining a monitoring configuration from memory means, the monitoring configuration including: a sensor configuration associated with the sensor set, a body profile associated with a subject body of the user and comprising reference data; providing, in memory means, measurement data relating to said subject body, said measurement data being acquired by said sensor set; computing, with computing means, a difference between the measurement data and the reference data; comparing, with comparison means, the difference and a predefined condition; generating a warning signal relating to a health level of the user, and/or a safety level of the user, with generating means, depending on the result of step comparing; and/or changing, with changing means, the monitoring configuration depending on the result of step comparing.

It is another aspect of the invention to provide a monitoring process, notably a user health and/or safety monitoring process, the monitoring process comprising the steps: providing a sensor set including at least one sensor; obtaining a monitoring configuration from memory means, the monitoring configuration including: a sensor configuration associated with the sensor set, a body profile associated with a subject body and comprising reference data; providing, in memory means, measurement data relating to said subject body, said measurement data being acquired by said sensor set; computing, with computing means, a difference between the measurement data and the reference data; comparing, with comparison means, the difference and a predefined condition; changing, with changing means, the monitoring configuration into an updated monitoring configuration depending on the result of step comparing; and monitoring the subject body depending on the updated monitoring configuration.

It is another aspect of the invention to provide a monitoring process, notably a user health and/or safety monitoring process, the monitoring process comprising the steps: providing a sensor set including at least one sensor; obtaining, from memory means, a monitoring configuration for computing a subject body monitoring data, the monitoring configuration including: a sensor configuration associated with the sensor set, a body profile associated with the subject body and comprising reference data; providing, in memory, measurement data relating to said subject body, said measurement data being acquired by said sensor set; computing, with computing means, a difference between the measurement data and the reference data; comparing, with comparison means, the difference and a predefined condition; changing, with changing means, the monitoring configuration depending on the result of step comparing.

It is another aspect of the invention to provide a computer program comprising computer readable code means, which when run on a computer, cause the computer to run the monitoring process according to the invention.

It is another aspect of the invention to provide a computer program product including a computer readable medium on which the computer program according to the invention.

It is another aspect of the invention to provide a computer configured for carrying out the monitoring process according to the invention.

It is another aspect of the invention to provide a wearable device configured for being worn by a user with a user body, the wearable device comprising a computer in accordance with the invention, the wearable device is preferably a shoe, a bracelet, a smart watch or a cellular phone. The different aspects of the invention may be combined to each other. In addition, the preferable features of each aspect of the invention may be combined with the other aspects of the invention, unless the contrary is explicitly mentioned.

### Technical Advantages of the Invention

The invention improves trustworthiness in monitoring. It avoids false warning signals since the model used for assessing the sensors and the user health or safety level is more suitable for the current situation. On top of this the system automatically reconfigures when a sensor is out of order. The use of machine learning and deep learning improves the analysis accuracy, which is of prior relevance since a typical sensor brings thousands of measures as day. As a corollary, a group with hundreds of users; each with several wearing devices; provides a big data.

The invention also optimises experience sharing. If a user, through its wearable device, detects an anormal situation and adopts a specific update, then this update is shared with a whole group, or a sub group, with a similar profile or a similar sensor set.

### BRIEF DESCRIPTION OF THE DRAWINGS

Several embodiments of the present invention are illustrated by way of figures, which do not limit the scope of the invention, wherein:

FIG. 1 provides a schematic illustration of a monitoring system for a sensor and a body in accordance with a preferred embodiment of the invention;

FIG. 2 provides a schematic illustration of a monitoring system for a sensor and a group of bodies in accordance with a preferred embodiment of the invention;

FIG. 3 provides a schematic illustration of a monitoring system for a sensors and sub groups of bodies in accordance with a preferred embodiment of the invention;

FIG. 4 provides a schematic illustration of a monitoring process in accordance with a first preferred embodiment of the invention;

FIG. 5 provides a schematic illustration of a monitoring process in accordance with a second preferred embodiment of the invention.

### DETAILED DESCRIPTION OF THE INVENTION

This section describes the invention in further detail based on preferred embodiments and on the figures. Similar reference numbers will be used to describe similar or the same concepts throughout different embodiments of the invention.

It should be noted that features described for a specific embodiment described herein may be combined with the features of other embodiments unless the contrary is explicitly mentioned.

Features commonly known in the art will not be explicitly mentioned for the sake of focusing on the features that are specific to the invention. For example, the monitoring system in accordance with the invention is evidently powered by an electric supply, even though such supply is not explicitly referenced on the figures nor referenced in the description.

In the current description, the term “wearable” means that it may be carried by a human being which is an average adult. It may be understood as having a weight of at most: 15 kg; or 5 kg; or 1 kg, or 200 g; or 100 g.

The wearable device in accordance with the invention may be wireless, or connected. When connected with a wire, for communication or power supply for instance, the weight of the wearable device is measured without said wire.

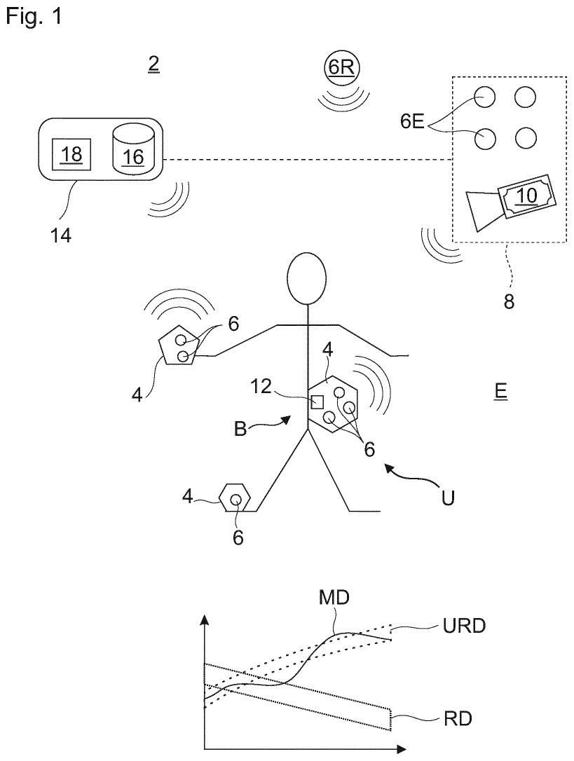

FIG. 1 shows a monitoring system 2 for a user U. The user U wears a wearable device 4 in accordance with the invention. The user U is represented in an environment E.

The user U is a human being. The user U is alive. The user U comprises a subject body B.

The user U is provided with at least one wearable device 4, and optionally comprises several wearable devices 4. The wearable devices 4 are at different locations of its subject body B.

The user U carries a first wearable device 4 at his arm, or in his hand. This first wearable device 4 may be a cellular phone, also designated as smartphone. It may also be a bracelet around one of its wrists. The user U is also equipped with a second wearable device 4. The second wearable device 4 is at the trunk of the subject body B. It may be at the waistline of the subject body B. The second wearable device 4 may be integrated into a belt, or an item of clothing. The used U is optionally furnished with a third wearable device 4. The third wearable device 4 is attached to the leg of the body B. It may be at a foot. The third wearable device 4 may be integrated into a shoe (not represented).

The user U may receive a fourth wearable device 4 (not represented). The fourth wearable device may be located at the head of the subject body B. The fourth wearable device may be housed in a head set or in a helmet.

As a further option, the user U may receive further wearable devices (not represented); for instance, at the symmetry of the firth four one as detailed above. By way of illustration, the user may receive a wearable device 4 at its left arm, and another one at the right arm.

The weight of the wearable devices represents at most: 5%, or 1%, or 0.2% of the weight of the subject body. Then, the motions of the user are less hindered. By way of illustration, the weight of the subject body is 75 kg.

At least one or each wearable device 4 comprises at least one sensor 6. The at least one wearable device may comprise several sensors 6. A computer program is associated with each sensor, notably for data transmission. Then, it is possible to obtain a piece of data. An aggregation software may be implemented. Multi-agent software platform may be used, where intelligent agents are connected to the actual probes forming the sensors. The agents are basically distributed over the main node of the system 2 composed mainly of sensors at the physical view of it. The agent-based architecture is the key on where to deploy three different types of agents. Different agent layers may be defined. The operational agent layer is associated with the metrics that will be considered and on top of which the probes are in charge of. The agents in the middle layer are responsible of aggregating the data coming from the operational agents; also designated as probe agents. The high-level agent layer is in charge of decision making.

The wearable device; or at least one associated sensor 6; is embedded or implanted in the subject body. It may be under the skin of the subject body.

The first to fourth wearable device 4 may comprise different number of sensors 6; some of them may comprise the same number of sensors which may; by way of illustration; range from one to three. The wearable devices 4 comprise power supplies, and communication means, for instance emitters configured for transmitting data from their sensors 6; or other data. As an option, the wearable devices 4 may comprise receiving means for receiving data; and notably for communication between them; and possibly with the environment. They communicate within the monitoring system 2.

The sensors 6 may be biometric sensors. The sensors 6 are adapted for measuring data relating to the health and/or a safety state of the user U. The sensors 6 may be configured for measuring or estimating the temperature of the body B of the user U. The sensors 6 may be adapted for providing the perspiration of the user body B. For this purpose, the corresponding sensor(s) may be adapted for measuring a humidity level on the skin of the subject body B of the user U. At least one sensor is adapted for measuring the heart rate of the user U.

As an option, the environment E comprises a device 8, which may be a fixed one with respect to the ground or the infrastructure in which the user U is. The device 8 may be wearable. It may be similar to the wearable devices 4. It comprises sensors, for instance environment sensors 6E.

The environment sensors 6 are configured for acquiring data relating to the environment E in which the user U moves. The environment sensor 6E are adapted for measuring at least one of the following: the temperature, the hygrometry, the air pressure, a sulphur content. Other environment data is considered by the current invention. The environment sensors 6E may comprise a camera 10.

The monitoring system 2 optionally includes a remote sensor 6R, at distance from the user U. The remote sensor 6R is at distance from the user U. It may be associated with a building, or with a vehicle (not represented) adapted for moving in the environment E. The remote senor 6R may capture sound and pictures form the user U. The remote sensor 6R may comprise a camera. The remote sensor 6R may be integrated in a flying drone.

The sensors 6 on the subject body, remote sensor 6R and the environment sensors 6E may be configured to provide the same kind of data. They may comprise the same electronic modules such as the camera, a microphone, a thermometer, a hygrometer, or a manometer adapted for measuring the environment pressure. Other modules are considered.

The sensors 6 attached to the subject body B define a sensor set. As an option the sensor set further comprises the environment sensors 6E and the remote sensor 6R. As an alternative, the sensor set only or at least comprise the remote sensor 6R; or the environment sensors 6E. A sensor set with a single sensor is considered through the current invention.

At least one or several wearable devices 4 comprise embedded computing means 12. The embedded computing means 12 are adapted for receiving a signal from one associated sensor 6. The embedded computing means 12 are adapted for computing data, storing data, and possibly communicate data; from the sensors (6; 6E; 6R).

As an alternative or in addition, the environment E exhibits centralized computing means 14. The centralized computing means 14 may communicate with the device 8, and with the wearable devices 4. It may communicate with the remote sensor 6R. As an option, the centralized computing means are in the device.

The computing means (12; 14) may communicate together. They each comprise a data storage unit 16, and at least one processor 18. The processors 18 are adapted for running computer program(s) in order to monitor the health and/or the safety of the user U. They may share computation steps, and may run in parallel. They may allow a distributed processing. The data storage units 18 are configured for storing a computer program code which is configured for, when run on the computing means (12, 14), cause the computing means to monitor the health and/or the safety of the user U.

Said computer program is adapted to cause the computing means (12, 14) to execute the monitoring process including the steps: providing a sensor set including at least one sensor; obtaining a monitoring configuration from memory means comprising at least one a memory element such as the data storage unit 16, the monitoring configuration including: a sensor configuration associated with the sensor set, a body profile associated with the subject body B and comprising reference data RD; providing, in a memory element, measurement data MD relating to said subject body B, said measurement data MD being acquired by said sensor (6; 6E; 6R) of the sensor set; computing, with the computing means (12; 14), a difference between the measurement data MD and the reference data RD; comparing, with comparison means formed by a module of the computing means (12; 14), the difference and a predefined condition; changing, with changing means, the monitoring configuration depending on the result of step comparing. The changing means may be formed by a module of the computing means (12; 14).

In the current embodiment a first monitoring configuration, also designated as initial monitoring condition, is changed into an updated monitoring configuration. Indeed, in the current measures the measurement data MD are remote and inconsistent with the reference data RD, which proves that the sensor configuration is not consistent with the current monitoring configuration. Another monitoring configuration more coherent must be selected out of a library of monitoring configurations with other body profiles. Said another monitoring configuration may be an updated monitoring data, also designated as replacement monitoring data, and may comprise an updated reference data URD corresponding to an updated body profile, also designated as second body profile. As an option, the sensor configuration becomes an updated sensor configuration which may be used for monitoring. The measurement data MD is closer to the updated reference data URD, and an updated difference is smaller than the previous one. The measurement data MD is more tailored to the updated reference data URD.

In the current FIG. 1, the measurement data MD is represented by means of a curve. However, the measuring data may comprise tables. Several graphs may be required for its representation. The measurement data MD may comprise time series.

Then, the invention avoids erroneous measuring configuration where a false dangerous situation is potentially detected. The tool for assessing safety and health is more in line with the user and the environment. The monitoring situation is corrected.

As an option or an alternative, the monitoring system 2 may comprise wires for connecting at least one wearable device 2. The wires may be used for providing a power supply, and/or for communication purposes.

The wearable device may be a wheelchair (not represented). The wheelchair comprises a seat place and several wheels, for instance at least three or four wheels. A frame joins the wheels to the seat place. The wheel chair is configured such that the subject body, respectively the user, actuates the wheels in order to drive and steer said wheel chair. As an option or an alternative, at least one; or the two front wheels; comprise in-wheel engines for driving or assisting the wheel drive and steering. At least one sensor or several sensors are arranged in the wheelchair. The weight of the wheelchair may range from 8 kg to 15 kg.

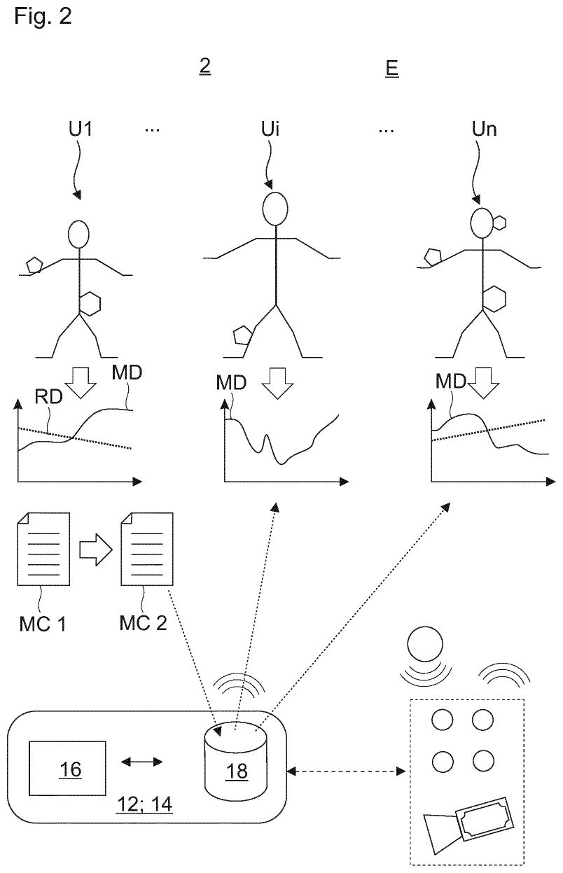

FIG. 2 shows a group of users Ui, said group comprising n users. The indicia i ranges from 1 to n, and n is a number of at least: 5, or 50 or 1000. The users Ui may be similar or identical to the user as described in relation with FIG. 1. The users Ui are associated with the monitoring system 2. The users Ui are in the environment E, which may be similar or identical to the environment as described throughout FIG. 1.

Each user Ui receives at least one wearable device and notably at least one sensor. The users Ui form a population. The users Ui comprise different numbers of wearable devices and/or of sensors. The numbers of wearable devices per user may vary from one to three; or more. Their wearable devices may be at different locations. As an alternative, all the users comprise the same wearable devices and/or sensors at the same locations. Same locations may be understood as on the same limb of their respective bodies. Each user Ui is associated with a subject body, and a body profile defined by a reference data.

These several users Ui comprise several wearable devices which form a first set of wearable devices, and a sensor sets; or “n” sensor sets. The number of wearable devices may vary from one user Ui to another one. One user Ui may comprise more wearable device than another user. During a measurement step, the sensors provides measurement data MD for each user Ui. With regard to the first subject body of the first user U1, the measurement data MD significantly deviates from the reference data RD. A difference can be computed in different ways between these data. The difference is computed by computing means (12; 14) which are centralized or enclosed in one of the wearable devices. At least one or several processors 18 may be used. The processors 18 are in a same computer unit, or distributed in a computer network.

Since the difference does not meet a predefined condition, the first monitoring configuration MC1 is changed into a second monitoring configuration MC2. In other words, the updated monitoring configuration MC2 replaces the initial monitoring configuration MC1, and brings a correction. The second monitoring configuration MC2 may comprises a second reference data, or a so-called updated reference data URD. The monitoring configuration provides data for comparison purpose.

The monitoring configuration comprises subject body monitoring data, which is used for assessing the health and/or the safety state.

The configuration change is stored in the data storage unit 16. Then, it may be communicated to the other users (U2-Un) which replaces their respective initial monitoring configuration by the second monitoring configuration MC2.

The change may by instantaneous. It may apply to a group of subject bodies in a same predefined area, for instance a manufacturing plant where a sulphur level may be part of the measurement data MD in order to trigger alarms.

As an alternative, the change may occur after a safety period during which it is observed whether a third monitoring configuration will be required due to another significant difference calculated with respect to the first user U1. If the second monitoring configuration MC2 proves its relevance and robustness over time, it is then implemented to the whole group.

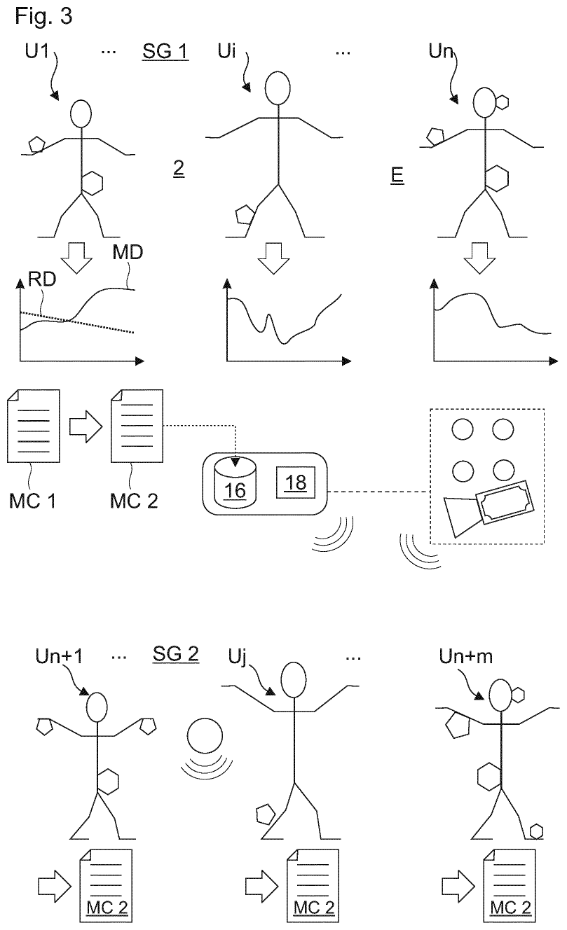

FIG. 3 shows a group of users forming a first sub group SG1 of users Ui and a second sub group SG2 of users Uj; where the indicia i varies from 1 to n; and the indicia j varies from n+1 to n+m. Other sub groups may be defined within this group. The users of the group and the environment E may be similar to the user and environment E as described in relation with FIG. 1. The sub groups may have different numbers of users (n; m).

Each user (Ui; Uj) receives at least one wearable device and notably at least one sensor. The users (Ui; Uj) comprise different numbers of wearable devices and/or of sensors. These numbers may vary within each sub groups (SG1; SG2). The numbers of wearable devices per user may vary from one to three; or more. Their wearable devices may be at different locations. As an alternative, all the users, for instance of one sub group, comprise the same wearable devices and/or sensors at the same locations. Each user (Ui; Uj) is associated with a subject body, and a body profile defined by a reference data.

The users Ui of the first sub group SG1 comprise several sensors which form a first set of sensors, or several first sensor sets. The users Uj of the second sub group SG comprise several wearable devices which form a second set of sensors devices, or several second sensor sets.

The number of wearable devices may vary from one user (Ui; Uj) to another one. One user Ui may comprise more wearable device than another user. Optionnally, one of the users U1 of the first sub group SG1 comprises the same number of sensors, and/or the same body profile, than the users Uj of the second sub group SG2. As a further option, one of the users U1 of the first sub group SG1 comprises the same monitoring configuration than the users Uj of the second sub group SG2.

During a measurement step; or observation step; the sensors provide measurement data MD for each user (Ui; Uj). With regard to the first subject body of the first user U1, the measurement data MD significantly deviates from the reference data RD. A difference can be computed in different manner between these data. The difference is computed by computing means (12; 14) which are centralized or enclosed in one of the wearable devices, for instance of users of each sub groups. At least one or several processors 18 may be used.

Since the difference meets a predefined condition, for instance a predefined threshold and/or a logic test, the first monitoring configuration MC1 is amended into a second monitoring configuration MC2. The new configuration is recorded in the data storage unit 16. Then, it may be transmitted to the users (Un+l-Um) of the second sub group SG2. This last monitoring configuration replaces the previous monitoring configuration of the subject bodies of the second sub group SG2.

The sub groups (SG1; SG2) may be defined on the basis of their body profiles, and/or of their sensors. In addition, the sub groups (SG1; SG2) may be defined by means of the history of their wearable devices. The system 2 accesses their gallery data, and analyses their pictures for instance.

By means of a deep learning model, specific features are identified amongst those pictures. Automatic classification provides and defines sub groups out of a population. In addition, the system 2 is also configured for analysing text from users. This text may be automatically generated on the basis of pictures, sounds records, or videos; thus, a multimedia content more generally. This text may be further obtained from other users (Ui; Uj) answering or commenting on the multimedia content.

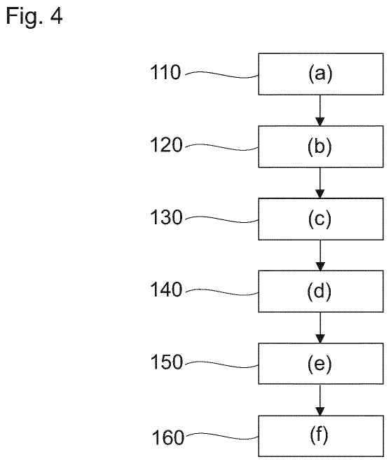

FIG. 4 provides a schematic illustration of a diagram illustrating the monitoring process in accordance with a first embodiment of the invention. The monitoring process is notably a sensor monitoring process, and/or a user monitoring process in order to assess a safety level or a health state of the user. The user may be similar as the one as described in relation with FIG. 1.

The monitoring process comprises the following steps, optionally executed as listed below:

(a) providing 110 a sensor set including at least one sensor;

(b) obtaining 120 a monitoring configuration from a memory element, the monitoring configuration including: a sensor configuration associated with the sensor set, a body profile associated with a subject body and comprising reference data;

(c) providing 130, in a memory element, measurement data relating to said subject body, said measurement data being acquired by said sensor set;

(d) computing 140, with computing means, a difference between the measurement data and the reference data;

(e) comparing 150, with comparison means, the difference and a predefined condition;

(f) changing 160, with changing means, the monitoring configuration depending on the result of step comparing.

The current monitoring process allows anormal measuring and monitoring configurations. It replaces the monitoring configuration which makes sense in view of the measurement data. Thus, a realistic monitoring may be carried out. The update of the monitoring configuration may comprise a replacement of a sensors, or more precisely rendering a first sensor in a disabled state, whereas a second sensor becomes enabled. These sensors swap their status.

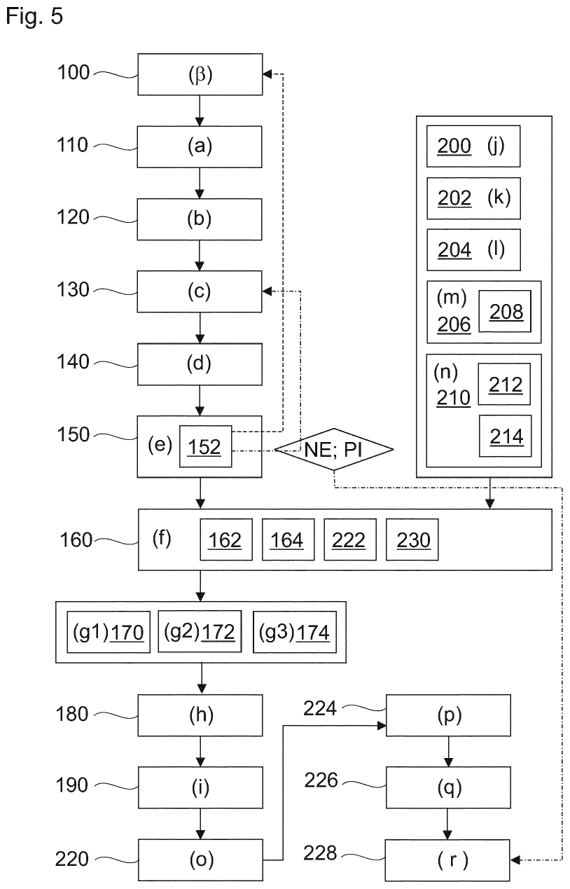

FIG. 5 provides a schematic illustration of a diagram illustrating the monitoring process in accordance with a second embodiment of the invention. The monitoring process is adapted for monitoring the health and/or the safety of at least one user, of a group of users, or of a sub group of users. The users are in accordance with the user as described in FIG. 1. The group and sub groups are in accordance with the one as described in FIGS. 2 and 3. The monitoring process is similar to the one as described in relation with FIG. 4.

The monitoring process comprises the following steps, optionally executed in the subsequent order:

(ß) training 100 a neuronal network;

(a) providing 110 a sensor set including at least one sensor;

(b) obtaining 120 a monitoring configuration from a memory element, the monitoring configuration including: a sensor configuration associated with the sensor set, a body profile associated with a subject body and comprising reference data;

(c) providing 130, in a memory element, measurement data relating to said subject body, said measurement data being acquired by said sensor set;

(d) computing 140, with computing means, a difference between the measurement data and the reference data;

(e) comparing 150, with comparison means, the difference and a predefined condition;

(f) changing 160, with changing means, the monitoring configuration depending on the result of step comparing;

(g1) generating 170, with generating means, a configuration change signal,

(g2) transmitting 172, with transmitting means, the configuration change signal, and

(g3) receiving 174, with receiving means, the configuration change signal;

(h) generating 180 a warning signal indicating that the subject body is in an unhealthy state or an unsafe state, notably in light of the updated monitoring configuration.

At step (a) providing 110 measurement data, the at least one sensor is physically in contact of the subject body and is adapted for measuring at least one of the following: a temperature of the subject body, a humidity level on the subject body, a heart rate of the subject body, and a blood pressure of the subject body. Their combinations are considered as well by the present invention. The humidity level comprises a perspiration on the skin of the subject body.

At step (b) obtaining 120, the body profile comprises at least one of the following: the weight of the subject body, the size of the subject body, the age of the subject body, and an average physical activity of the subject body. The body profile is optionally defined in relation with a predefined area.

For instance, in an hospital a reference data of the body profile may be adapted to this specific place. Here, people are intended to stay in a rest state. Their heart rate, their temperature, their perspiration is intended to remain low. By contrast, when a user is climbing a stair, he his making a specific effort. Then, the reference data range may be enlarged before a monitoring configuration change is technically required. A high heart rate would be locally acceptable. Then, the invention adapts the body profile locally and/or upon the physical activity, thus the body motion. The context is taken into account.

Step (c) providing 130 measurement data comprises a sub-step measuring 132 said measurement data by means of the sensor set. As an alternative, the measurement data is obtained from a data storage which provides past measurements.

At step (d) computing 140 the difference, weight parameters are applied to pieces of data of the measurement data, and are computed by means of at least one of the following: a subject body temperature, a subject body physical activity, and a subject body perspiration information. The weight parameters are applied in order to reduce the impact of several pieces of data, and to increase the effect of other ones.

At step (e) comparing 150, the predefined condition comprises a predefined threshold, at step comparing the difference meets the predefined condition provided that the measurement data diverges from the reference data by the predefined threshold. The difference may be computed by means of a ratio, a subtraction, a polynomic. Derivative computation and statistics may be implemented.

The monitoring process comprises a step capturing or obtaining 152 environment information, at step (e) comparing 150 the difference between the measurement data and the reference data is computed with a neuronal network model adapted for detecting anormal monitoring configurations by means of said environment information. The neuronal network model is optionally trained at step (ß) training 100, or further trained at a subsequent iteration of the monitoring process.

At step (f) changing 160 the monitoring configuration is changed into an updated monitoring configuration and the first subject body is in a predefined area, which may be a factory, or a retirement house.

Afterward, the monitoring process further comprises a step (o) changing 220 a monitoring configuration of another subject body which is in said predefined area, into the updated monitoring configuration. The first subject body and the other subject body may be in the predefined area at different times. The configuration change may be stored in the centralized monitoring means, and provided to the other subject body when it enters the predefined area of interest.

At step (f) changing 160 the monitoring configuration, the process comprises a step capturing or obtaining 222 environment information, said environment information feeding a learning database, or picture history. Then, the learning database gathers anormal monitoring configurations. The learning database is used for training a neuronal network at step ((3) training 100 the neuronal network in order to compute said neuronal network model.

The step (f) changing 160 may comprise, or be replaced by a step of correcting 160 the monitoring configuration. When the monitoring configuration deviates from a standard configuration, is switched into a corrected configuration.

At step (f) changing 160 the monitoring configuration is changed in accordance with the configuration change signal. Then, the monitoring configuration is changed into an updated monitoring configuration. The process comprises a step updating 230, or step (f) changing 160 comprises a sub step updating 230, the monitoring configuration into an updated monitoring configuration.

At step (g2) transmitting 172, the transmission of the data is done over the distributed agent-based architecture to consolidate the situation for a given context. Here, one user is associated with one environment. It may be considered, depending from the use case, that such a system can be deployed to many users within different contexts. This becomes a source of data on top of which analytics can be considered; such that the system can make accurate and personalised for each user in each use case. In the current context of information transmission, all communication means are considered. For instance, internet protocols-based means via smart phone applications, IoT and M2M based communication means are enclosed in the current invention.

The feedback link communication to the users once a decision is taken can be done via a worldwide network. Messages on personalised spaces may be used, direct messages via phones using communication means such as cellular 2G; 3G; 4G; 5G protocols are considered as well. Any communication means such as those by satellite are considered.

Step (g3) receiving 174 may be mainly achieved by the monitoring agent also considered as a high-level agent, which is in charge of providing the decision via a cockpit-based visualisation solution. For illustrative purpose, several signal may be provided. These signals may be a sub step: alerting on the system set-up, alerting on the quality of the sensors themselves, alerting on the safety of the users.

At step (h) generating 180, if the signals coming directly from the sensors are only checked, it may be concluded on a normal functioning of the system, assuming by simplicity that the current analysis relies on the positive behaviours of the sensors in isolation. In the current approach, different metrics are defined in service-based approach. Defining metrics to be monitored is in charge for instance to capture a correlation risk in combining data coming from different sensors and come up to identify potential failures which might come up with a warning signal.

The reference data is a first reference data, the difference is a first difference, at step (b) obtaining 120 the monitoring configuration, the body profile is a first body profile of a profile library further comprising a second body profile with a second reference data. Step (f) changing 160 the monitoring configuration optionally encloses a sub-step replacing 162 the first body profile by the second body profile and a step computing 164 a second difference between the measurement data and the second reference data. This may be repeated several times with a third reference data, a fourth reference data and so on until a suitable body profile is identified. Step computing 164 a second difference may be a sub step, and an independent step.

By contrast with the first difference, the second difference does not meet said predefined condition. This implies that this profile update introduces another body profile which is consistent with the measured data. This solves a problem where there is a mistake in the body profile: for instance, the age of the user is unrealistic.

By way of illustration, a profile may correspond to an age of 80 years. By convention, the reference data may enclose a maximum heart rate of 140 ppm, or 100 ppm depending on the safety required. A predefined condition comprises a heart rate difference of at most 50 ppm. A user which is 15 years old may select this profile by mistake. During an effort period, this user reaches a heart rate of 230 ppm; which may be considered as impossible to reach for an 80 years old user. If the user aged of 15 provides a heart rate of 230 ppm whereas he has selected a profile with an age of 80, the difference between the reference heart rate and the measured heart rate exceeds the predefined condition. Then, there is an anomalous situation, the body profile is changed, thus the monitoring configuration is changed. The change may comprise an automatic selection of a profile corresponding to user with an age comprised between 10 and 20 years. Other data may be used. Different data may be combined in order to identify the true body profile. Further, if the measurement data provides a heart rate of 20 ppm, it implies that the currently used sensor is not reliable. Another one must replace it. The profile may be refined iteratively over time.

Step (h) generating 180 is triggered provided that at step (b) obtaining 120 the reference data comprises a reference range, and the body profile comprises a rest state data with a rest value within a reference range. If the measurement data enclose a piece of data in the reference range, for instance a heart rate within a rest heart rate range; and the difference does not meet the predefined condition, the monitoring process comprises the step (h) generating 180 a warning signal. This warning signal is obtained when the configuration is realistic, and the user is in a real hazardous situation.

The predefined condition comprises a first environment threshold, a first body information threshold, which is associated with the first environment threshold. The monitoring process comprises a step (i) providing 190 environment information and a body information, and further comprising said step (f) changing 160 provided that said environment information exceeds the first environment threshold and the body information is below the first body information threshold. As an alternative the first body information threshold is replaced by a body reference range which is associated with the first environment threshold.

The monitoring process further comprises a step (j) providing 200 a body temperature and an environment temperature, and comprising step (f) changing 160 the monitoring configuration provided that the environment temperature exceeds the first temperature threshold and the body temperature does not reach a second temperature threshold associated with the first temperature threshold.

The monitoring process further comprises a step (k) providing 202 a body perspiration information and an environment hygrometry, and actually comprising step (f) changing 160 the monitoring configuration depending on hygrometry and perspiration. If the environment hygrometry exceeds a first hygrometry threshold and the body perspiration information is below the first perspiration threshold associated with the first hygrometry threshold, the monitoring process executes step (f) changing 160 the monitoring configuration.

The predefined condition comprises a third temperature threshold and a second perspiration threshold, associated with the third temperature threshold. The monitoring process further comprises a step (l) providing 204 an environment temperature and a body perspiration information. The monitoring process actually triggers (f) changing 160 the monitoring configuration provided that the environment temperature exceeds the third temperature threshold and the body perspiration information is below the second perspiration threshold.

If the body perspiration is too low in light of the hygrometry and of the environment temperature, it is concluded that there is an anomaly in the monitoring configuration.

The predefined condition comprises a second body information threshold and a motion threshold, the monitoring process further comprises a step (m) tracking 206 motion information of the subject body, and a step providing 208 body information. The current body information includes, in a non-limitative way, the heart rate, the subject body temperature, the subject body perspiration. The monitoring process actually comprising step (f) changing 160 the monitoring configuration provided that the body information is below the second body information threshold and the motion information exceeds the motion threshold. The motion threshold may be a speed threshold or an acceleration threshold. If the body moves by his own of a predefined distance during a predefined period, the threshold may be reached. Then the monitoring configuration is anormal, and must be adapted.

The monitoring process further comprises a step (n) identifying 210 a neighbour subject body with a neighbour sensor set. The later includes at least one sensor of a sensor set; a neighbour body profile defined by a neighbour reference data. The distance between the first subject body and the neighbour subject body is smaller than a threshold distance which implies that these bodies are close to one another at the time of step (n) identifying 210. The monitoring process further comprises a step acquiring 212 neighbour measurement data relating to the neighbour subject body; a step computing 214 a neighbour difference between the neighbour measurement data and the neighbour reference data. The monitoring process comprising said step (f) changing 160 the monitoring configuration provided that the neighbour difference diverges from the predefined condition, or meet other requirements, the requirement and the predefined condition being mutually exclusive.

As an option, the first body profile and the neighbour body profile are identical, the first subject body and the neighbour subject body comprising different sensor configurations. As an alternative, the first subject body and the neighbour subject body comprises identical sensor configurations, whereas the first body profile differs from the neighbour body profile. Thus, the first body profile and the neighbour body profile differ on one aspect, but share common parameter(s).

The subject body is a first subject body which is part of a group of subject bodies, each subject body being associated with a sensor set including at least one sensor; a monitoring configuration with a sensor configuration associated and a body profile comprising a reference data. The sensor set may be identical throughout the group. The monitoring process further comprises a step (p) defining 224, within said group, a first sub group of subject bodies which comprise similar or identical monitoring configurations, said first sub group being defined by means of a neural network model configured for defining sub groups on the basis of their measurement data, the first subject body being part of said first sub group.

At step (0 changing 160 the monitoring configuration of the first subject body receives an updated monitoring configuration. Then, the monitoring process comprises a step (q) changing 226 monitoring configurations of the other subject bodies of the first sub group into said updated monitoring configuration. This phenomenon is detailed in relation with FIG. 2 with respect to a group, and with a sub group at FIG. 3.

The monitoring process repeats steps (a) providing 110, (d) computing 140 and (e) comparing 150 while a predefined period PE and/or a predefined number of iterations NI. If these steps (110; 140; 150 are repeated without triggering step (0 changing 160 the monitoring configuration again, then the monitoring process comprises a step (r) changing 228 the monitoring configuration of a third subject body into said updated monitoring configuration. It may be thereby understood that if the adopted monitoring configuration is stable because it is not changed again during a significant period, then it is proven that this monitoring configuration is suitable to the corresponding user and/or sensor set.

Features detailed in relation with a user may also apply to a subject body associated with said user; and vice versa.

It should be understood that the detailed description of specific preferred embodiments is given by way of illustration only, since various changes and modifications within the scope of the invention will be apparent to the person skilled in the art. The scope of protection is defined by the following set of claims.

1. A monitoring process for monitoring at least one of a user health and a user safety, the monitoring process comprising the steps:
providing a sensor set including at least one sensor;
obtaining a monitoring configuration from memory means, the monitoring configuration including:
a sensor configuration associated with the sensor set,
a body profile associated with a subject body and comprising reference data;
providing, in memory means, measurement data relating to said subject body, said measurement data being acquired, by said sensor set;
computing, with computing means, a difference between the measurement data and the reference data;
comparing, with comparison means, the difference and a predefined condition; and
changing, with changing means, at least one of the sensor configuration and the body profile of the monitoring configuration, depending on a result of the comparing step.

2. The monitoring process in accordance with claim 1, wherein, provided that at the step of comparing the difference meets the predefined condition, the monitoring process further comprises the steps:
generating, with generating means, a configuration change signal;
transmitting, with transmitting means, the configuration change signal; and
transmitting, with receiving means, the configuration change signal, wherein at the step of changing, the monitoring configuration is changed in accordance with the configuration change signal.

3. The monitoring process in accordance with claim 2, wherein the receiving means are comprised in the sensor set.

4. The monitoring process in accordance with claim 1, wherein the predefined condition comprises a predefined threshold, at the step of comparing the difference meets the predefined condition provided that the measurement data diverges from the reference data by, at least, the predefined threshold.

5. The monitoring process in accordance with claim 1, wherein the reference data comprises a reference range, and the body profile comprises a rest state with a rest value within the reference range.

6. The monitoring process in accordance with claim 5, wherein if the measurement data comprises a piece of data in the reference range, and the difference does not meet the predefined condition, the monitoring process comprises a step generating a warning signal indicating that the subject body is at least one of in an unhealthy stand and in an unsafe state.

7. The monitoring process in accordance with claim 1, wherein the at least one sensor is a first sensor and at the step of providing the measurement data, the first sensor is enabled, and wherein at the step of changing the monitoring configuration the first sensor is disabled.

8. The monitoring process in accordance with claim 1, wherein the sensor set further comprises a second sensor and at the step of providing the measurement data, the second sensor is disabled, and wherein at the step of changing the monitoring configuration, the second sensor is enabled.

9. The monitoring process in accordance with claim 1, wherein the step of providing the measurement data comprises a sub-step measuring said measurement data by means of the sensor set.

10. The monitoring process in accordance with claim 1, wherein the reference data is a first reference data, the difference is a first difference, wherein at the step of obtaining the monitoring configuration, the body profile is a first body profile of a profile library further comprising a second body profile with a second reference data, the step of changing the monitoring configuration comprising a sub-step replacing the first body profile by the second body profile, the monitoring process further comprising a step computing a second difference between the measurement data and the second reference data, the first difference meeting the predefined condition and the second difference does not meet said predefined condition.

11. The monitoring process in accordance with claim 1, wherein the predefined condition comprises at least one of a first environment threshold, a first body information threshold and, a body reference range, which are associated with the first environment threshold, wherein the monitoring process comprising a step providing environment information and a body information, and comprising said step of changing the monitoring configuration provided that said environment information exceeds the first environment threshold and the body information is below at least one of the first body information threshold and the body reference range.

12. The monitoring process in accordance with claim 1, wherein the predefined condition comprises a first temperature threshold and a second temperature threshold, associated with the first temperature threshold, the monitoring process comprising a step providing a body temperature and an environment temperature, and comprising said step of changing the monitoring configuration provided that the environment temperature exceeds the first temperature threshold and the body temperature is below the second temperature threshold.

13. The monitoring process in accordance with claim 1, wherein the predefined condition comprises a first hygrometry threshold and a first perspiration threshold, associated with the first hygrometry threshold, the monitoring process comprising a step providing a body perspiration information and an environment hygrometry, and comprising said step of changing the monitoring configuration provided that the environment hygrometry exceeds the first hygrometry threshold and the body perspiration information is below the first perspiration threshold.

14. The monitoring process in accordance with claim 1, wherein the predefined condition comprises a third temperature threshold and a second perspiration threshold, associated with the third temperature threshold, the monitoring process comprising a step providing an environment temperature and a body perspiration information, and comprising said step of changing the monitoring configuration provided that the environment temperature exceeds the third temperature threshold and the body perspiration information is below the second perspiration threshold.

15. The monitoring process in accordance with claim 1, wherein the predefined condition comprises a second body information threshold and a motion threshold, the monitoring process further comprises a step tracking motion information of the subject body, and a step providing a body information such as a heart rate, a subject body temperature, a subject body perspiration, the monitoring process comprising said step of changing the monitoring configuration provided that the body information is below the second body information threshold and the motion information exceeds the motion threshold.

16. The monitoring process in accordance with claim 1, wherein at the step of computing the difference, weight parameters are applied to pieces of data of the measurement data, said weight parameters being computed by means of at least one of the following: a subject body temperature, a subject body physical activity, and a subject body perspiration information.

17. The monitoring process in accordance with claim 1, wherein the subject body is a first subject body, the body profile is a first body profile, the measurement data is a first measurement data, the monitoring process further comprising a step identifying a neighbour subject body with a neighbour sensor set including at least one sensor, a neighbour body profile defined by a neighbour reference data, the distance between the first subject body and the neighbour subject body being smaller than a threshold distance, a step acquiring neighbour measurement data relating to the neighbour subject body, the monitoring process further comprising a step computing a neighbour difference between the neighbour measurement data and the neighbour reference data, the monitoring process comprising said step of changing provided that the neighbour difference does not meet the predefined condition.

18. The monitoring process in accordance with claim 17, wherein the first body profile and the neighbour body profile are identical, the first subject body and the neighbour subject body comprising different sensor configurations.

19. The monitoring process in accordance with claim 1, wherein at the step of changing the monitoring configuration the monitoring configuration is changed into an updated monitoring configuration and the subject body is in a predefined area, the monitoring process further comprising a step changing a monitoring configuration, into said updated monitoring configuration, of another subject body-body which is in said predefined area.

20. The monitoring process in accordance with claim 1, wherein the monitoring process comprises a step of capturing environment information, at the step of comparing the difference between the measurement data and the reference data is computed with a neuronal network model adapted for detecting anormal monitoring configurations by means of said environment information.

21. The monitoring process in accordance with claim 20, wherein at the step of changing the monitoring configuration, the process comprises the step of capturing the environment information, said environment information feeding a learning database of anormal monitoring configurations, the process further comprising a step training a neuronal network by means of the learning database in order to compute said neuronal network model.

22. The monitoring process in accordance with claim 1, wherein the subject body is a first subject body which is part of a group of subject bodies, each subject body being associated with a sensor set including at least one sensor, a monitoring configuration with a sensor configuration associated and a body profile comprising a reference data, the monitoring process comprising a step defining, within said group, a first sub group of subject bodies which comprise identical monitoring configurations, said first sub group being defined by means of a neural network model configured for defining sub groups on the basis of their measurement data, the first subject body being part of said first sub group.

23. The monitoring process in accordance with claim 22, wherein at the step of changing the monitoring configuration, the monitoring configuration of the first subject body is changed into an updated monitoring configuration, the monitoring process comprises a step changing monitoring configurations of the other subject bodies of the first sub group into said updated monitoring configuration.

24. The monitoring process in accordance with claim 1, wherein the monitoring process comprises a step updating the monitoring configuration into an updated monitoring configuration, the monitoring process repeating the step of providing the measurement data, the step of computing the difference and the step of comparing at least one of the difference during a predefined period and a predefined number of iterations during which the difference does not meet the predefined condition, then the monitoring process further comprises a step changing the monitoring configuration of a third subject body into said updated monitoring configuration.

25. The monitoring process in accordance with claim 1, wherein the sensor set comprises at least one biometric sensors adapted for measuring biometric data of said subject body.

26. The monitoring process in accordance with claim 1, wherein the sensor set comprises at least one environment sensor adapted for acquiring an information of the environment of the subject body, said at least one environment sensor comprising at least one of the following: a camera, a microphone, a thermometer, a hygrometer, and a manometer adapted for measuring an environment pressure.

27. The monitoring process in accordance with claim 1, wherein the sensor set further comprises a remote sensor at distance from the subject body, said remote sensor being fixed to a ground-based infrastructure with respect to which the subject body moves during the monitoring process.

28. The monitoring process in accordance with claim 1, wherein at the step of providing measurement data, the at least one sensor is physically in contact of the subject body and is adapted for measuring at least one of the following: a temperature of the subject body, a humidity level on the subject body, a heart rate of the subject body, and a blood pressure of the subject body, the humidity level being a perspiration on a skin of the subject body.

29. The monitoring process in accordance with claim 1, wherein the body profile comprises at least one of the following: a weight of the subject body, a size of the subject body, an age of the subject body, and an average physical activity of the subject body.

30. The monitoring process in accordance with claim 1, wherein the subject body is a human subject body.

31. A computer program comprising computer readable code means, which when the program is executed by a computer, cause the computer to carry out the monitoring process according to claim 1.

32. A computer program product including a computer readable medium on which the computer program according to claim 31 is stored.

33. A computer configured for carrying out the monitoring process according to claim 1.

34. A wearable device configured for being worn by a user with a user body, the wearable device comprising a computer in accordance with claim 33; the wearable device is at least one of a shoe, a bracelet, a smart watch, and a cellular phone.
